(19) United States
(12) Patent Application Publication
(10) Pub. No.: US 2025/0152529 A1
(43) Pub. Date: May 15, 2025

(54) METHOD OF ESCALATING SOTALOL HYDROCHLORIDE DOSING

(71) Applicant: AltaThera Pharmaceuticals LLC, Chicago, IL (US)

(72) Inventors: John Charin Somberg, Chicago, IL (US); Brandon Ira Kashfian, Chicago, IL (US); Janos Molnar, Chicago, IL (US)

(21) Appl. No.: 19/019,556

(22) Filed: Jan. 14, 2025

Related U.S. Application Data
(63) Continuation of application No. 17/725,189, filed on Apr. 20, 2022.
Application No. 17/725,189 is a continuation of application No. 16/946,941, filed on Jul. 13, 2020, now Pat. No. 11,344,518.
Application No. 16/946,941 is a continuation-in-part of application No. 16/863,567, filed on Apr. 30, 2020, now abandoned.
Application No. 16/946,941 is a continuation-in-part of application No. 16/849,099, filed on Apr. 15, 2020, now abandoned.
(60) Provisional application No. 63/009,511, filed on Apr. 14, 2020.
(60) Provisional application No. 62/987,832, filed on Mar. 10, 2020.

(51) Int. Cl. A61K 31/18; A61K 9/00; A61P 9/06

(52) U.S. Cl. CPC A61K 31/18; A61K 9/0019; A61P 9/06

(57) ABSTRACT

The present invention provides novel methods of initiating and escalating sotalol hydrochloride in patients in need thereof.

### CROSS-REFERENCE TO RELATED APPLICATION

This application is a Continuation application of U.S. application Ser. No. 17/725,189, filed Apr. 20, 2022, which application is a Continuation application of U.S. application Ser. No. 16/946,941, filed Jul. 13, 2020, which issued as U.S. Pat. No. 11,344,518 on May 31, 2022. The '941 application is a Continuation-in-Part application of U.S. application Ser. No. 16/863,567 filed Apr. 30, 2020, which '567 application claims priority to and the benefit of the filing date of U.S. Provisional Application No. 63/009,511, filed Apr. 14, 2020. The '941 application is a Continuation-in-Part application of U.S. application Ser. No. 16/849,099, filed Apr. 15, 2020, which '099 application claims priority to and the benefit of the filing date of U.S. Provisional Application No. 62/987,832 filed Mar. 10, 2020. These applications are hereby incorporated by reference herein in their entireties.

### FIELD OF THE INVENTION

The present invention provides novel methods of initiating and escalating sotalol hydrochloride in patients in need thereof.

### BACKGROUND OF THE INVENTION

Sotalol hydrochloride (“sotalol”) is Vaughan Williams Class III anti-arrhythmic drug that prolongs cardiac action potential duration by blocking the outward potassium channel IKr (rapid potassium rectifier current), thereby prolonging repolarization time.

Although sotalol is effective at treating or preventing atrial fibrillation, atrial flutter, and combinations thereof, sotalol's mechanism of action is both anti-arrhythmic and pro-arrhythmic. Too much sotalol too fast can lead to excessive prolongation of repolarization time giving rise to life threatening arrhythmias, especially Torsade de Pointes ventricular tachycardia (Tdp). Thus, it is well understood in the art, that during sotalol's initial loading, or in a dose escalation intervention, it is critical to monitor a subject's QTc interval to avoid excessive QTc prolongation.

Due to sotalol's potential to induce arrhythmia, the FDA has mandated in-hospital QTc monitoring for at least three days upon initial sotalol hydrochloride loading and for dose escalation. Although this extended hospital stay is effective at reducing subject risk, maintaining the subject in a telemetry unit for three days is extremely expensive, making sotalol a less desirable treatment choice in a managed care environment. A need therefore exists to develop methods of reducing the length of hospital stay required to safely and efficaciously administer sotalol to subjects in need thereof.

### SUMMARY OF THE INVENTION

Accordingly, in an aspect, the present invention provides a novel method of initiating or escalating sotalol hydrochloride in a patient in need thereof.

In another aspect, the present invention provides a method of reducing the hospitalization time required for initiating and escalating sotalol hydrochloride in a patient in need thereof.

In another aspect, the present invention provides a method of treating AFIB/AFL, comprising: initiating or escalating sotalol hydrochloride in a patient in need thereof.

These and other aspects, which will become apparent during the following detailed description, have been achieved by the inventors' discovery that oral sotalol can be intravenously initiated or escalated.

### DETAILED DESCRIPTION OF THE INVENTION

All references cited herein are hereby incorporated in their entirety herein by reference.
AFIB is atrial fibrillation.
AFL is atrial flutter.
AFIB/AFL=atrial fibrillation and/or atrial flutter.
IV is intravenous.
PO means “per os” and refers to an oral dosing regimen.
BID means “bis in die” and means twice a day.
QD means “quaque die” and means once a day.
Patient (or subject) refers to a human patient.
BP is blood pressure.
HR is heart rate.

Renally impaired refers to patients having creatine clearance rates of ≤60 mL/min.

Escalation or uptitration means increasing the oral sotalol dosage of a patient (e.g., from 80 to 120 or from 120 to 160 BID).

QT is the interval measured from the start of the Q wave or the QRS complex, to the end of the T wave, where the Q wave corresponds to the beginning of ventricular depolarization and the T wave end corresponds to the end of ventricular repolarization.

QTc is the calculated interval that represents the QT interval corrected for heart rate and can be derived by simple mathematical correlation of the QT interval and the heart rate.

AQTc is the difference between a QTc measurement taken prior to the start of IV sotalol and a QTc measured after the start of IV sotalol (e.g., during loading or maintenance).

Sotalol and sotalol hydrochloride (used interchangeably herein) refer to d, l-sotalol hydrochloride which has been approved by the FDA for intravenous administration over 5 hours or oral administration (e.g., 80 mg, 120 mg, and 160 mg tablets).

In an example, sotalol injection is supplied in 10 mL single-dose vials, each containing 150 mg of sotalol hydrochloride as a clear solution (15 mg/mL).

Sotalol hydrochloride is contraindicated in patients with:
Sinus bradycardia (<50 bpm), sick sinus syndrome or second or third degree AV block without a pacemaker.
Congenital or acquired long QT syndromes, QT interval >450 ms
Cardiogenic shock, decompensated heart failure.
Serum potassium <4 mEq/L.
Bronchial asthma or related bronchospastic conditions
Known hypersensitivity to sotalol

Hospital refers to a medical facility staffed and equipped to provide continuous ECG monitoring and cardiac resuscitation to patients, if needed. Typically, the medical personnel are trained in the management of serious ventricular arrhythmias.

Reducing or shortening the length of a hospital stay refers to reducing/shortening the length of time a patient is admitted for oral sotalol initiation or escalation. For example, a patient would typically require a 3-day (72 hour) stay to be initiated/escalated on oral sotalol.

The terms “treat,” “treating,” and “treatment” refer to any indicia of success in the treatment or amelioration of an injury, disease, or condition, including any objective or subjective parameter such as abatement; remission; diminishing of symptoms or making the injury, disease, or condition more tolerable to the subject; slowing in the rate of degeneration or decline; or improving a subject's physical or mental well-being. The treatment or amelioration of symptoms can be based on objective or subject parameters, including the results of a physical examination, neuropsychiatric examinations, or psychiatric evaluation.

### Sotalol Indications:

Delay in Recurrence of Atrial Fibrillation/Atrial Flutter: Sotalol is indicated for the maintenance of normal sinus rhythm (delay in time to recurrence of atrial fibrillation/atrial flutter (AFIB/AFL)) in patients with symptomatic AFIB/AFL who are currently in sinus rhythm.

Life-Threatening Ventricular Arrhythmia: Sotalol is also indicated for the treatment of life-threatening ventricular tachycardia.

For either indication, intravenous sotalol, when used as a loading dose, achieves steady state concentration faster compared to the conventional oral dosing (e.g., typically 3-days for a non-renally impaired patient).

Typically, IV sotalol is diluted for infusion. For example, IV sotalol can be diluted in saline, 5% dextrose in water (D5W), or Ringer's lactate. The dilution volume chosen is one that is convenient for administration and consistent with fluid restriction. A volumetric infusion pump can be used to administer the IV sotalol.

Typically, other antiarrhythmic therapy is withdrawn prior to starting sotalol.

The IV loading dose depends on the target oral dose and creatinine clearance of the patient indicated for oral sotalol. The dosing interval for oral administration of sotalol and the minimum delay between the end of the infusion and the first oral dose also depend on renal function.

Thus, in an aspect, the present invention provides a novel method of initiating or escalating sotalol therapy, comprising: administering to a patient in need thereof (a patient for whom sotalol is indicated) an IV and oral sotalol dosage based on the patient's creatine clearance (CrCl mL/min), wherein the dosages and timing are selected from Table 1.

TABLE 1
IV Sotalol Loading Dosage and Initiation-Escalation Protocol

| | IV loading dose (1 h infusion) | | | | | |
|---|---|---|---|---|---|---|
| | when the oral dose is going from . . . | | | | | |
| Creatinine | Sotalol Initiation | | Sotalol Escalation | | Minimum | |
| Clearance* | 0 to 80 | 0 to 120 | 80 to 120 | 120 to 160 | delay to first | Oral dosing |
| (mL/min) | mg** | mg | mg | mg | oral dose (h) | interval (h) |
| >90 | 60 | 90 | 75 | 90 | 4 | 12 |
| >60-90 | 82.5 | 125 | 82.5 | 105 | 4 | 12 |
| >30-60 | 75 | 112.5 | 82.5 | 105 | 6 | 24 |
| 10-30 | 75 | 112.5 | 82.5 | 105 | 12 | 48 |

*Calculated using Cockcroft-Gault formula
**Recommended starting dose

The Cockcroft-Gault formulas for creatine clearance (CrCl) are:

$$CrCl ( male ) = ( ( 140 - age ) \times weight in kg ) / ( serum creatinine \times 72 )$$

$$CrCl ( female ) = CrCl ( male ) \times 0.85$$

Recommended starting dose (80 mg) is the FDA recommended dosage. A physician can select to start a patient on a higher dosage (e.g., 120 mg), if deemed appropriate.

Minimum delay to first oral dose is the time from the end of the IV infusion to the first oral dose.

Oral dosing interval refers to the time between oral dosages. 12 h is B.I.D. (or BID). 24 h is Q.D. (or QD).

The IV load, as shown in Table 1, is typically administered (infused) over 1 hour. Additional examples include 50-70 minutes. Further examples include 50, 51, 52, 53, 54, 55, 56, 57, 58, 59, 60, 61, 62, 63, 64, 65, 66, 67, 68, 69, and 70 minutes.

Examples of the IV load for a target oral dose of 80 mg include 55-85 mg. For example, Table 1 shows that the IV loads for initiation of a target dose of 80 mg are 60 mg (>90 mL/min CrCl), 82.5 mg (60-90 mL/min CrCl), and 75 mg (30-60 mL/min CrCl and 10-30 mL/min CrCl). Further examples include 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, and 85 mg.

Examples of the IV load for a target oral dose of 120 mg include 75-115 mg. For example, Table 1 shows that the IV loads for initiation of a target dose of 120 mg are 90 mg (>90 mL/min CrCl), 125 mg (60-90 mL/min CrCl), and 112.5 mg (30-60 mL/min CrCl and 10-30 mL/min CrCl). Additional examples of the IV load for the target dose of 120 mg include 75-135 mg. Further examples include 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, and 135 mg.

Further, for example, Table 1 shows that the IV loads for escalation from 80 to 120 mg are 75 mg (>90 mL/min CrCl), and 82.5 (60-90 mL/min CrCl, 30-60 CrCl, and 10-30 mL/min CrCl). Additional examples of the IV load for escalation to 120 mg include 65-90 mg. Further examples include 65, 66, 67, 68, 69, 70, 71, 72, 73, 74, 75, 76, 77, 78, 79, 80, 81, 82, 83, 84, 85, 86, 87, 88, 89, and 90 mg.

Examples of the IV load for a target oral dose of 160 mg include 80-150 mg. For example, Table 1 shows that the IV loads for escalation from 120 to 160 mg are 90 mg (>90 mL/min CrCl), and 105 (60-90 mL/min CrCl, 30-60 mL/min CrCl, and 10-30 mL/min CrCl). Additional examples of the IV load for escalation to 160 mg include 80-120 mg. Further examples include 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 and 150 mg.

As can be seen in Table 1, the time of when oral dosing begins depends on the CrCl of the patient. Oral dosing for CrCl of >90 mL/min and 60-90 mL/min typically begins 4 h after IV infusion (e.g., 5 hours after the start of a 1 h IV). Oral dosing for CrCl of 30-60 mL/min typically begins 6 h after IV infusion (e.g., 7 h after the start of a 1 h IV). Oral dosing for CrCl of 10-30 mL/min typically begins 12 h after IV infusion (e.g., 13 h after the start of a 1 h IV).

Additional examples of when the oral dosing begins for a CrCl of >90 mL/min include 2-6 h after completion of infusion. Further examples include 2, 3, 4, 5, to 6 h.

Additional examples of when the oral dosing begins for a CrCl of 60-90 mL/min include 2-6 h after completion of infusion. Further examples include 2, 3, 4, 5, to 6 h.

Additional examples of when the oral dosing begins for a CrCl of 30-60 mL/min include 4-8 h after completion of infusion. Further examples include 4, 5, 6, 7, to 8 h.

Additional examples of when the oral dosing begins for a CrCl of 10-30 mL/min include 10-14 h after completion of infusion. Further examples include 10, 11, 12, 13, to 14 h.

As can be seen in Table 1, the oral dosing interval depends on the CrCl of the patient. Oral dosing for CrCl of >90 mL/min and 60-90 mL/min is typically every 12 h (BID). Oral dosing for CrCl of 30-60 mL/min is typically every 24 H (QID). Oral dosing for CrCl of 10-30 mL/min is typically every 48 h.

Additional examples of the oral dosing interval for a CrCl of >90 mL/min include 8-16 h after completion of infusion. Further examples include 8, 9, 10, 11, 12, 13, 14, 15, to 16 h.

Additional examples of the oral dosing interval for a CrCl of 60-90 mL/min include 8-16 h after completion of infusion. Further examples include 8, 9, 10, 11, 12, 13, 14, 15, to 16 h.

Additional examples of the oral dosing interval for a CrCl of 30-60 mL/min include 20-28 h after completion of infusion. Further examples include 20, 21, 22, 23, 24, 25, 26, 27, to 28 h.

Additional examples of when the oral dosing begins for a CrCl of 10-30 mL/min include 40-56 h after completion of infusion. Further examples include 40, 41, 42, 43, 44, 45, 46, 47, 48, 49, 50, 51, 52, 53, 54, 55, to 56 h.

In another aspect, the present invention provides a novel method of initiating or escalating sotalol therapy, comprising: administering to a patient in need thereof an IV and oral dosage of sotalol selected by a physician, wherein the patient's physician selects the IV and oral dosages based on the patient's CrCl as defined in Table 1.

In another aspect, the present invention provides a novel method of initiating or escalating oral sotalol therapy, comprising:

A intravenously (IV) administering sotalol hydrochloride over a period of 1 hour, to a patient in need thereof, wherein the IV dosage is selected from dosages I (a)-IV (d):
I for patients having creating clearance (CrCl) of >90 mL/min;
   (a) 60 mg for a patient naïve to sotalol and having a target of 80 mg;
   (b) 90 mg for a patient naïve to sotalol and having a target of 120 mg;
   (c) 75 mg for a patient previously receiving 80 mg of sotalol;
   (d) 90 mg for a patient previously receiving 120 mg of sotalol;
II for patients having creating clearance (CrCl) of >60-90 mL/min;
   (a) 82.5 mg for a patient naïve to sotalol and having a target of 80 mg;
   (b) 125 mg for a patient naïve to sotalol and having a target of 120 mg;
   (c) 82.5 mg for a patient previously receiving 80 mg of sotalol;
   (d) 105 mg for a patient previously receiving 120 mg of sotalol;
III for patients having creating clearance (CrCl) of >30-60 mL/min;
   (a) 75 mg for a patient naïve to sotalol and having a target of 80 mg;
   (b) 112.5 mg for a patient naïve to sotalol and having a target of 120 mg;
   (c) 82.5 mg for a patient previously receiving 80 mg of sotalol;
   (d) 105 mg for a patient previously receiving 120 mg of sotalol;
IV for patients having creating clearance (CrCl) of 10-30 mL/min;
   (a) 75 mg for a patient naïve to sotalol and having a target of 80 mg;
   (b) 112.5 mg for a patient naïve to sotalol and having a target of 120 mg;
   (c) 82.5 mg for a patient previously receiving 80 mg of sotalol; and,
   (d) 105 mg for a patient previously receiving 120 mg of sotalol; and,

B orally administering sotalol hydrochloride to the patient at a dosage and interval selected from (I)-(IX):
I 80 mg BID, starting 4 hours after completion of IV doses I (a) and II (a);
II 120 mg BID, starting 4 hours after completion of IV doses I (b), I (c), II (b), and II (c);
III 160 mg BID, starting 4 hours after completion of IV doses I (d) and II (d);
IV 80 mg QD, starting 6 hours after completion of IV dose III (a);
V 120 mg QD, starting 6 hours after completion of IV doses III (b) and III (c);
VI 160 mg QD, starting 6 hours after completion of IV dose III (d);
VII 80 mg once every 48 h, starting 12 hours after completion of IV dose IV (a);
VIII 120 mg once every 48 h, starting 12 hours after completion of IV doses IV (b) and IV (c); and,
IX 160 mg once every 48 h, starting 12 hours after completion of IV dose IV (d).

In another aspect, the present invention provides a novel method of initiating or escalating oral sotalol therapy, comprising:

A intravenously (IV) administering sotalol hydrochloride over a period of 1 hour, to a patient in need thereof, wherein the IV dosage is selected from dosages I (a)-IV (d):
I for patients having creating clearance (CrCl) of >90 mL/min;
   (a) 60 mg for a patient naïve to sotalol and having a target of 80 mg;
   (b) 90 mg for a patient naïve to sotalol and having a target of 120 mg;
   (c) 75 mg for a patient previously receiving 80 mg of sotalol;
   (d) 90 mg for a patient previously receiving 120 mg of sotalol;
II for patients having creating clearance (CrCl) of >60-90 mL/min;
   (a) 82.5 mg for a patient naïve to sotalol and having a target of 80 mg;
   (b) 125 mg for a patient naïve to sotalol and having a target of 120 mg;
   (c) 82.5 mg for a patient previously receiving 80 mg of sotalol;
   (d) 105 mg for a patient previously receiving 120 mg of sotalol;
III for patients having creating clearance (CrCl) of >30-60 mL/min;
   (a) 75 mg for a patient naïve to sotalol and having a target of 80 mg;
   (b) 112.5 mg for a patient naïve to sotalol and having a target of 120 mg;
   (c) 82.5 mg for a patient previously receiving 80 mg of sotalol;
   (d) 105 mg for a patient previously receiving 120 mg of sotalol;
IV for patients having creating clearance (CrCl) of 10-30 mL/min;
   (a) 75 mg for a patient naïve to sotalol and having a target of 80 mg;
   (b) 112.5 mg for a patient naïve to sotalol and having a target of 120 mg;
   (c) 82.5 mg for a patient previously receiving 80 mg of sotalol; and,
   (d) 105 mg for a patient previously receiving 120 mg of sotalol; and,

B orally administering sotalol hydrochloride to the patient at a dosage and interval selected from (I)-(IX):
I 80 mg BID, starting 3-5 hours after completion of IV doses I (a) and II (a);
II 120 mg BID, starting 3-5 hours after completion of IV doses I (b), I (c), II (b), and II (c);
III 160 mg BID, starting 3-5 hours after completion of IV doses I (d) and II (d);
IV 80 mg QD, starting 5-7 hours after completion of IV dose III (a);
V 120 mg QD, starting 5-7 hours after completion of IV doses III (b) and III (c);
VI 160 mg QD, starting 5-7 hours after completion of IV dose III (d);
VII 80 mg once every 48 h, starting 10-14 hours after completion of IV dose IV (a);
VIII 120 mg once every 48 h, starting 10-14 hours after completion of IV doses IV (b) and IV (c); and,
IX 160 mg once every 48 h, starting 10-14 hours after completion of IV dose IV (d).

In another aspect, the present invention provides a novel method of initiating or escalating oral sotalol therapy, comprising:

A intravenously (IV) administering sotalol hydrochloride over a period of 1 hour, to a patient in need thereof, wherein the IV dosage is selected from dosages I (a)-IV (d):
I for patients having creating clearance (CrCl) of >90 mL/min;
   (a) 50-70 mg for a patient naïve to sotalol and having a target of 80 mg;
   (b) 80-100 mg for a patient naïve to sotalol and having a target of 120 mg;
   (c) 65-85 mg for a patient previously receiving 80 mg of sotalol;
   (d) 80-100 mg for a patient previously receiving 120 mg of sotalol;
II for patients having creating clearance (CrCl) of >60-90 mL/min;
   (a) 72.5-92.5 mg for a patient naïve to sotalol and having a target of 80 mg;
   (b) 115-135 mg for a patient naïve to sotalol and having a target of 120 mg;
   (c) 72.5-92.5 mg for a patient previously receiving 80 mg of sotalol;
   (d) 95-115 mg for a patient previously receiving 120 mg of sotalol;
III for patients having creating clearance (CrCl) of >30-60 mL/min;
   (a) 65-85 mg for a patient naïve to sotalol and having a target of 80 mg;
   (b) 102.5-122.5 mg for a patient naïve to sotalol and having a target of 120 mg;
   (c) 72.5-92.5 mg for a patient previously receiving 80 mg of sotalol;
   (d) 95-115 mg for a patient previously receiving 120 mg of sotalol;
IV for patients having creating clearance (CrCl) of 10-30 mL/min;
   (a) 65-85 mg for a patient naïve to sotalol and having a target of 80 mg;
   (b) 102.5-122.5 mg for a patient naïve to sotalol and having a target of 120 mg;
   (c) 72.5-92.5 mg for a patient previously receiving 80 mg of sotalol; and,
   (d) 95-115 mg for a patient previously receiving 120 mg of sotalol; and,

B orally administering sotalol hydrochloride to the patient at a dosage and interval selected from (I)-(IX):
I 80 mg BID, starting 4 hours after completion of IV doses I (a) and II (a);
II 120 mg BID, starting 4 hours after completion of IV doses I (b), I (c), II (b), and II (c);
III 160 mg BID, starting 4 hours after completion of IV doses I (d) and II (d);
IV 80 mg QD, starting 6 hours after completion of IV dose III (a);
V 120 mg QD, starting 6 hours after completion of IV doses III (b) and III (c);
VI 160 mg QD, starting 6 hours after completion of IV dose III (d);
VII 80 mg once every 48 h, starting 12 hours after completion of IV dose IV (a);
VIII 120 mg once every 48 h, starting 12 hours after completion of IV doses IV (b) and IV (c); and,
IX 160 mg once every 48 h, starting 12 hours after completion of IV dose IV (d).

In another aspect, the present invention provides a novel method of initiating or escalating oral sotalol therapy, comprising:

A intravenously (IV) administering sotalol hydrochloride over a period of 40-80 minutes, to a patient in need thereof, wherein the IV dosage is selected from dosages I (a)-IV (d):
I for patients having creating clearance (CrCl) of >90 mL/min;
   (a) 50-70 mg for a patient naïve to sotalol and having a target of 80 mg;
   (b) 80-100 mg for a patient naïve to sotalol and having a target of 120 mg;
   (c) 65-85 mg for a patient previously receiving 80 mg of sotalol;
   (d) 80-100 mg for a patient previously receiving 120 mg of sotalol;
II for patients having creating clearance (CrCl) of >60-90 mL/min;
   (a) 72.5-92.5 mg for a patient naïve to sotalol and having a target of 80 mg;
   (b) 115-135 mg for a patient naïve to sotalol and having a target of 120 mg;
   (c) 72.5-92.5 mg for a patient previously receiving 80 mg of sotalol;
   (d) 95-115 mg for a patient previously receiving 120 mg of sotalol;
III for patients having creating clearance (CrCl) of >30-60 mL/min;
   (a) 65-85 mg for a patient naïve to sotalol and having a target of 80 mg;
   (b) 102.5-122.5 mg for a patient naïve to sotalol and having a target of 120 mg;
   (c) 72.5-92.5 mg for a patient previously receiving 80 mg of sotalol;
   (d) 95-115 mg for a patient previously receiving 120 mg of sotalol;
IV for patients having creating clearance (CrCl) of 10-30 mL/min;
   (a) 65-85 mg for a patient naïve to sotalol and having a target of 80 mg;
   (b) 102.5-122.5 mg for a patient naïve to sotalol and having a target of 120 mg;
   (c) 72.5-92.5 mg for a patient previously receiving 80 mg of sotalol; and,
   (d) 95-115 mg for a patient previously receiving 120 mg of sotalol; and,

B orally administering sotalol hydrochloride to the patient at a dosage and interval selected from (I)-(IX):
I 80 mg BID, starting 3-5 hours after completion of IV doses I (a) and II (a);
II 120 mg BID, starting 3-5 hours after completion of IV doses I (b), I (c), II (b), and II (c);
III 160 mg BID, starting 3-5 hours after completion of IV doses I (d) and II (d);
IV 80 mg QD, starting 5-7 hours after completion of IV dose III (a);
V 120 mg QD, starting 5-7 hours after completion of IV doses III (b) and III (c);
VI 160 mg QD, starting 5-7 hours after completion of IV dose III (d);
VII 80 mg once every 48 h, starting 10-14 hours after completion of IV dose IV (a);
VIII 120 mg once every 48 h, starting 10-14 hours after completion of IV doses IV (b) and IV (c); and,
IX 160 mg once every 48 h, starting 10-14 hours after completion of IV dose IV (d).

In another aspect, the patient is indicated for chronic, but not acute sotalol therapy.

In another aspect, the patient is under ECG monitoring.

In another aspect, the patient is in a hospital for the IV loading and at least 2 oral dosages.

In another aspect, the patient experiences 3 sotalol steady state Cₘₐₓ in less than 24 hours, which allows for a QTc interval of the subject that corresponds to the full concentration effect of sotalol to be assessed in less than 24 hours (as opposed to the typical ˜72 hours required for oral initiation and/or escalation).

In another aspect, the patient is considered to have been initiated/escalated on oral sotalol after having experienced 3 sotalol steady state Cₘₐₓ.

In a patient having a CrCl of >60 mL, at least 5-6 doses of oral sotalol (BID) are recommended before the patient is able to be discharged. In a patient having a CrCl of 40-60 mL, at least 5-6 doses of oral sotalol (QD) are recommended before the patient is able to be discharged. Currently, patients with a CrCl <40 mL/min are not recommended for oral sotalol.

In another aspect, the patient is considered to have been initiated/escalated on oral sotalol after having received at least at least 2, 3, or 4 oral dosages.

In another aspect, the patient is considered to have been initiated/escalated on oral sotalol after having received at least at least 2 oral dosages and the patient's CrCl is >90 mL/min. In another example, the patient received 3 oral dosages. In another example, the patient received 4 oral dosages.

In another aspect, the patient is considered to have been initiated/escalated on oral sotalol after having received at least at least 2 oral dosages and the patient's CrCl is >60-90 mL/min. In another example, the patient received 3 oral dosages. In another example, the patient received 4 oral dosages.

In another aspect, the patient is considered to have been initiated/escalated on oral sotalol after having received at least at least 2 oral dosages and the patient's CrCl is >30-60 mL/min. In another example, the patient received 3 oral dosages. In another example, the patient received 4 oral dosages.

In another aspect, the patient is considered to have been initiated/escalated on oral sotalol after having received at least at least 2 oral dosages and the patient's CrCl is 10-30 mL/min. In another example, the patient received 3 oral dosages. In another example, the patient received 4 oral dosages.

In another aspect, the method of initiating or escalating oral sotalol therapy, further comprises: prior to IV loading, measuring the patient's serum creatine level and calculating the patient's Creatine Clearance (CrCl) using the Cockcroft-Gault formula.

In another aspect, the patient's baseline QTc is measured prior to initiation or escalation.

In another aspect, only patients having a QTc <450 ms are initiated or escalated.

In another aspect, the patient's QTc interval is measured every 15 minutes during IV infusion.

In another aspect, the patient's QTc interval is measured for 2-4 hours after the first oral dosage.

In another aspect, the patient's QTc interval is measured for 2-4 hours after the second oral dosage for patients having a CrCl of ≥60 mL/min.

In another aspect, if the patient's QTc interval is >500 ms or if the AQTc is 20% when initiating an 80 mg oral dosage, the method is discontinued.

In another aspect, the present invention provides a method of treating AFIB/AFL, comprising: initiating or escalating sotalol hydrochloride as described herein in a patient in need thereof (e.g., see Table 1).

In another aspect, the method reduces the overall time of hospitalization of the patient (compared to a patient initiated or escalated with only oral sotalol).

In another aspect, the patient being initiated/escalated on sotalol and having a CrCl of >60 mL/min is able to be discharged from the hospital in less than the standard 72 hours required for only oral sotalol initiation/escalation. For example, the patent is able to be discharged (or is discharged) from 18-48 hours after IV initiation. Additional examples include from 18-36 hours, from 18-24 hours, and from 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, to 48 hours after IV initiation.

In another aspect, the patient being initiated/escalated on sotalol and having a CrCl of from 10-60 mL/min is able to be discharged from the hospital in less than the standard 5-6 days (5-6 dosages QD) required for oral-only sotalol initiation/escalation and having a CrCl of 40-60 mL/min. For example, the patent is able to be discharged (or is discharged) from 24-96 hours after IV initiation. Additional examples include from 24-72 hours, from 36-48 hours, and from 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, to 72 hours after IV initiation.

In another aspect, the oral therapy is maintained at the patient's physician's discretion. For example, the oral therapy can be continued for days, weeks, or months.

In another aspect, the patient's QTc is monitored via electrocardiography.

In another aspect, the patient's QTc is measured at baseline (prior to sotalol administration) and then measured periodically thereafter (e.g., every 15 or 30 minutes during loading). The QTc can be measured at other intervals if more (shorter time period) or less data (longer time periods) data is desired.

In another aspect, the HR and BP the patient is monitored every 15 minutes (or 30 minutes) during IV administration. If a BP below 90 mmHg and HR <50 bpm are observed, then the IV is discontinued. The HR and BP are also typically monitored for 15-30 minutes after completion of the IV administration.

In another aspect, the present invention provides a novel pharmaceutical composition, comprising: a syringe, comprising: from 60-125 mg of sotalol. In another example, the syringe consists essentially of 60-125 mg of sotalol and at least one diluent selected from: saline, 5% dextrose in water (D5W), and Ringer's lactate.

In another aspect, the syringe, comprises: 60 mg of sotalol. In another example, the syringe consists essentially of 60 mg of sotalol and at least one diluent selected from: saline, 5% dextrose in water (D5W), and Ringer's lactate.

In another aspect, the syringe, comprises: 75 mg of sotalol. In another example, the syringe consists essentially of 75 mg of sotalol and at least one diluent selected from: saline, 5% dextrose in water (D5W), and Ringer's lactate.

In another aspect, the syringe, comprises: 82.5 mg of sotalol. In another example, the syringe consists essentially of 82.5 mg of sotalol and at least one diluent selected from: saline, 5% dextrose in water (D5W), and Ringer's lactate.

In another aspect, the syringe, comprises: 90 mg of sotalol. In another example, the syringe consists essentially of 90 mg of sotalol and at least one diluent selected from: saline, 5% dextrose in water (D5W), and Ringer's lactate.

In another aspect, the syringe, comprises: 105 mg of sotalol. In another example, the syringe consists essentially of 105 mg of sotalol and at least one diluent selected from: saline, 5% dextrose in water (D5W), and Ringer's lactate.

In another aspect, the syringe, comprises: 112.5 mg of sotalol. In another example, the syringe consists essentially of 112.5 mg of sotalol and at least one diluent selected from: saline, 5% dextrose in water (D5W), and Ringer's lactate.

In another aspect, the syringe, comprises: 125 mg of sotalol. In another example, the syringe consists essentially of 125 mg of sotalol and at least one diluent selected from: saline, 5% dextrose in water (D5W), and Ringer's lactate.

In another aspect, the volume of liquid in the syringe is from 5, 6, 7, 8, 9, to 10 mL.

In another aspect, the syringe is filled from a vial of 15 mg/mL of sotalol and further diluted to a final volume of 10 mL, wherein the diluent is selected from, saline, 5% dextrose in water (D5W), and Ringer's lactate.

The present invention may be embodied in other specific forms without departing from the spirit or essential attributes thereof. This invention encompasses all combinations of aspects of the invention noted herein. It is understood that any and all embodiments of the present invention may be taken in conjunction with any other embodiment or embodiments to describe additional embodiments. It is also to be understood that each individual element of the embodiments is intended to be taken individually as its own independent embodiment. Furthermore, any element of an embodiment is meant to be combined with any and all other elements from any embodiment to describe an additional embodiment.

Other features of the invention will become apparent in the course of the following descriptions of exemplary embodiments that are given for illustration of the invention and are not intended to be limiting thereof.

### EXAMPLES

### Example Sotalol Protocol

Infuse the loading dose over one hour.
Monitor QTc interval every 15 minutes during infusion.
Continue to monitor QTc around Tmax (2 to 4 hours post-dose) following the first oral dose (in all patients) and second oral dose (in patients with CrCl ≥60 mL/min).
If the QTc interval prolongs to >500 ms or increases 20% from baseline when initiating for an oral dose of 80 mg, discontinue drug; if initiating for an oral dose of 120 mg discontinue drug and consider a lower dose. If re-initiation at a lower dose of 80 mg is desired, wait at least 1 day (in patients with CrCl ≥60 mL/min), or at least 3 days (in patients with CrCl ≥30 to <60 mL/min), or 7 days (in patients with CrCl ≥10 to <30 mL/min).

Numerous modifications and variations of the present invention are possible considering the above teachings. It is therefore to be understood that within the scope of the appended claims, the invention may be practiced otherwise that as specifically described herein.

1. A method of escalating sotalol hydrochloride, dosing comprising:
a. escalating an existing sotalol concentration of a subject to a higher level by administering an intravenous (IV) dose of 105 mg sotalol hydrochloride over a period of 1 hour to the subject, wherein the subject had symptomatic atrial fibrillation or atrial flutter and is currently in sinus rhythm; and
b. after completion of the IV dose, administering oral dosing of one or more oral dose of sotalol hydrochloride to the subject in an amount of 160 mg;
wherein the subject is discharged from the hospital 24 hours after IV initiation.

2. The method of claim 1, wherein the oral dosing is initiated 4-12 hours after completion of the IV dose.

3. (canceled)

4. The method of claim 1, wherein the oral dosing is initiated 4-6 hours after completion of the IV dose.

5. The method of claim 1, wherein the oral dosing is initiated 6-12 hours after completion of the IV dose.

6. The method of claim 2, wherein one or more of the oral doses are administered at an interval of 12-48 hours from initiation of the oral dosing.

7. The method of claim 4, wherein one or more of the oral doses are administered at an interval of 12-24 hours from initiation of the oral dosing.

8. The method of claim 5, wherein one or more of the oral doses are administered at an interval of 24-48 hours from initiation of the oral dosing.

9. The method of claim 1, wherein:
the oral dosing is initiated 4 hours after completion of the IV dose; and
one or more of the oral doses are administered at an interval of 12 hours from initiation of the oral dosing.

10. The method of claim 1, wherein:
the oral dosing is initiated 6 hours after completion of the IV dose; and
one or more of the oral doses are administered at an interval of 24 hours from initiation of the oral dosing.

11. The method of claim 1, wherein:
the oral dosing is initiated 12 hours after completion of the IV dose; and
one or more of the oral doses are administered at an interval of 48 hours from initiation of the oral dosing.

12. The method of claim 1, wherein the subject is receiving 120 mg oral sotalol hydrochloride therapy and the IV dose is administered to escalate the existing sotalol concentration of the subject to a higher concentration before administering the 160 mg oral dose of sotalol hydrochloride.
